(19) United States
(12) Patent Application Publication
(10) Pub. No.: US 2024/0176598 A1
(43) Pub. Date: May 30, 2024

(54) SYSTEM AND METHOD FOR THE AUTOMATED DEVELOPMENT OF AI PROJECTS

(71) Applicant: MASTERCARD INTERNATIONAL INCORPORATED, Purchase, NY (US)

(72) Inventors: James Conway, Dublin (IE); Ngoc Minh Tran, Dublin (IE); Stephen Patrick Flinter, Dublin (IE)

(21) Appl. No.: 18/491,923

(22) Filed: Oct. 23, 2023

Related U.S. Application Data
(60) Provisional application No. 63/385,608, filed on Nov. 30, 2022.

(51) Int. Cl. G06F 8/35

(52) U.S. Cl. CPC G06F 8/35

(57) ABSTRACT

A system and method are provided for automating the development of artificial intelligence projects. The system includes a project store that enables the storage, searching and retrieval of existing artificial intelligence projects. The system also includes a data store that enables the storage, searching and retrieval of data sets. A search engine is provided that enables selected features to be applied to neural networks as part of the existing artificial intelligence projects. An artificial intelligence project server is also provided which utilizes a model training and validation module, a model interference module and an input module.

### CLAIM OF PRIORITY

This application claims priority to U.S. provisional application Ser. No. 63/385,608 filed Nov. 30, 2022. The disclosure of the priority application is incorporated by reference herein in its entirety.

### FIELD

This application relates to a system and method for the automated development of artificial intelligence (AI) projects and, more specifically, to a system and method for automatically leveraging existing AI projects to develop new AI projects.

### BACKGROUND

Multiple artificial intelligence projects often share a common pattern and, as a result, the features generated for these different projects share a common pattern.

What is needed is a system and method that leverages the features generated for previous AI projects with a common pattern for the development of new AI projects founded on the same common pattern. Such a solution will provide multiple benefits, including efficient development of new AI projects by leveraging existing AI projects, automated development of new AI projects, lowered maintenance and operation costs relating to the development and deployment of AI projects, and exploitation of complicated and dynamic features for AI projects save the time and cost needed to create new AI projects and improve the quality of resulting trained models.

### BRIEF DESCRIPTION OF THE DRAWINGS

The drawings described below are for illustrative purposes only and are not necessarily drawn to scale. The drawings are not intended to limit the scope of the disclosure in any way. Wherever possible, the same or like reference numbers are used throughout the drawings to refer to the same or like parts.

FIG. 1 is a diagram of a system for implementing a method for the automated development of AI projects according to the embodiments disclosed herein.

FIGS. 2A-2B features a flow diagram of a method for the automated development of AI projects according to the embodiments disclosed herein.

### DETAILED DESCRIPTION

As mentioned above, this application relates to a system and method for the automated development of artificial intelligence (AI) projects and, more specifically, to a method and system for automatically leveraging existing AI projects to develop new AI projects.

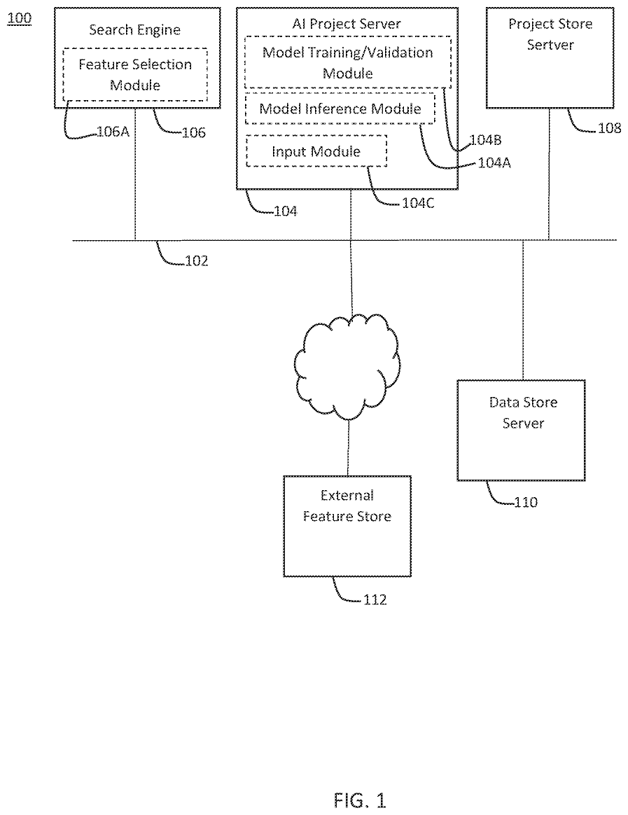

FIG. 1 is a diagram of a system 100 for implementing a method for the automated development of AI projects according to the embodiments disclosed herein. The system 100 may include a communications network 102 interconnecting an AI project server 104, a search engine 106, a project store server 108, a data store server 110, and an external feature store 112.

The network 102 may be a local area network (LAN), a wide area network (WAN), the Internet using one or more appropriate network or web services, or any other configuration known to one of ordinary skill in the art.

The search engine 106, the AI project server 104, the project store server 108, and the data store server 110 may each be a processor-based computing system capable of executing instructions. Moreover, each processor-based computing system may comprise a single computing machine or a collection of computing machines. A collection of computing machines may individually or jointly execute sets of instructions.

The project store server 108 may provide for the storage, searching, and retrieval of existing AI projects. Each existing AI project stored on the project store server 108 may include a detailed description of the existing AI project. Each existing AI project stored on the project store server 108 may further include accompanying models, feature engineering modules, and configuration parameters.

The data store server 110 may provide for the storage, searching, and retrieval of data sets, including data sets with a common pattern. These data sets may be used for training purposes in the development of new AI projects.

The search engine 106 is a processor-based engine that executes instructions that implement at least a feature selection module 106A providing for the manual or automated selection of features applied to neural networks as part of the existing AI projects. The feature selection module 106A implementing a forward feature selection method, a backward feature elimination method, or any other selection method known to one of ordinary skill in the art.

The search engine 106 further executing instructions that implement a grid search, random search, Bayesian search, or any other standard search method known to a person of ordinary skill in the art.

The AI project server 104 is a processor-based engine that executes instructions that implement a model training and validation module 104A, a model inference module 104B, and an input module 104C.

The model training and validation module 104A provides for the creation, training, and validation of neural network-based models within new AI projects. This model training and validation module 104A may implement a cross-validation method for the training and validation of the neural network-based models within new AI projects.

The model inference module 104B provides for serving over the communications network 102 of the neural network-based models that have been trained.

The input module 104C provides for receiving user inputs either directly or over the communication network 102. The input module 104C may provide a graphical user interface over the communication network 102.

The external feature store 112 provides model training services over the communication network 102, including predefined feature engineering modules. The external feature store 112 may be any third-party feature service provider known to a person of ordinary skill.

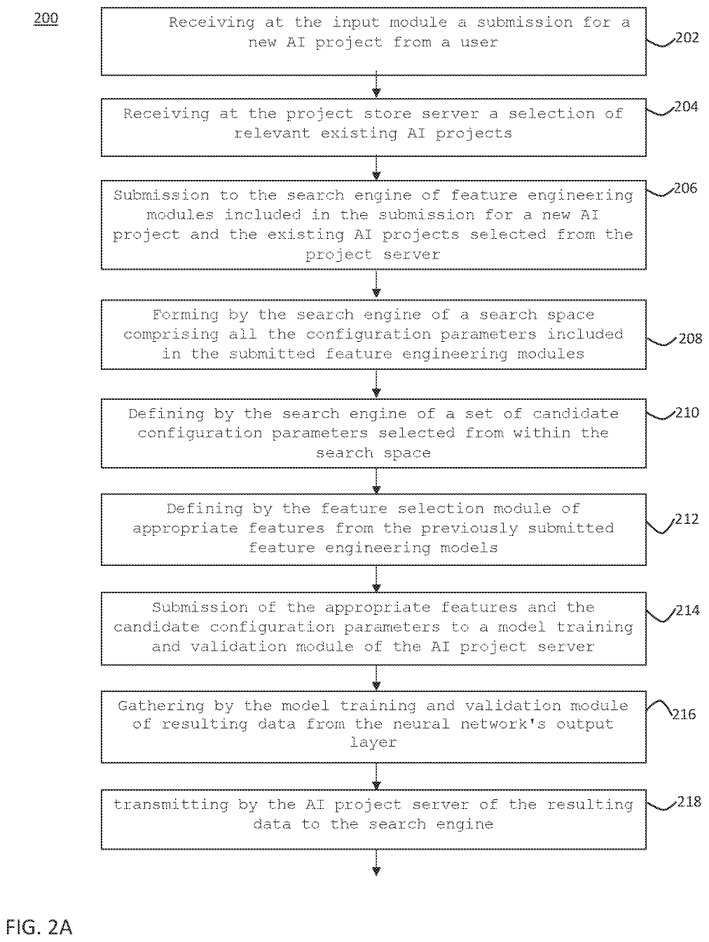

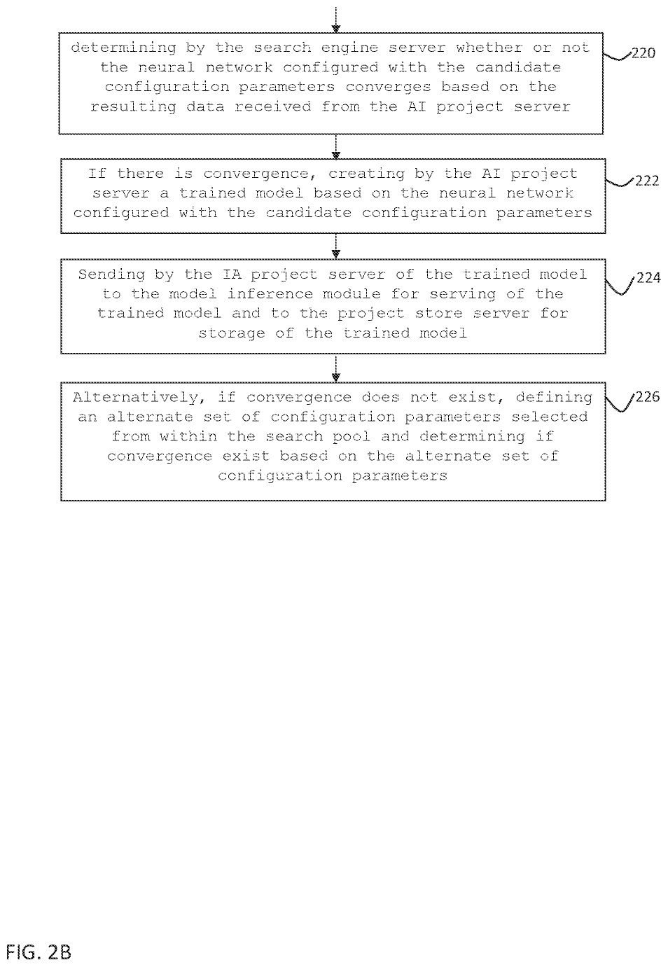

FIGS. 2A-2B features a flow diagram of a method 200 for the automated development of AI projects according to the embodiments disclosed herein.

The method 200 begins in step 202 with the input module 104C on the AI project server 104 receiving a submission for a new AI project from a user over the communication network 102.

The submission for a new AI project may include a feature engineering module for the new AI project. The submission may also include a text-based description that may be used to select existing AI projects.

The feature engineering module may include functions implementing preprocessing steps necessary to transform raw data into features for a machine-learning algorithm for the new AI project. For example, these preprocessing steps may include creating, transforming, extracting, and selecting features or variables most conducive to creating an accurate model by a machine learning algorithm. The functions may be implemented in a computer programming language reasonably known to one of the ordinary skill in the art.

The feature engineering module may further include a listing of variables derived from a raw data set, these variables being the inputs to the machine learning algorithm for the AI project.

The feature engineering module may further include configuration parameters that define the weights and biases associated with inputs to each node within a neural network. These weights and biases may be adjusted while optimizing the neural network's output, often through backpropagating updated versions of these configuration parameters.

In addition or as an alternative to including a feature engineering module in the submission for a new AI project, the user, in step 204, may select appropriate existing AI projects from the project store server 108.

As with a feature engineering module included in a submission, each existing AI project stored on the project store server 108 may or may not include a feature engineering module and text-based description. Similarly, each feature engineering module stored on the project store server 108 may or may not include corresponding configuration parameters.

In addition to the project store server 108, the user may select a feature engineering module from the external feature store 112.

The existing AI projects stored on the project store server 108 may all have been trained using raw data stored on the data store server 110 having a common pattern or domain space.

In one exemplary embodiment, the sets of raw data stored on the data store server 110 may all have a common pattern reflective of merchant transactions. In this exemplary embodiment, the feature engineering modules stored on the project store server 108 may all have features corresponding to this common pattern reflective of merchant transactions.

In another exemplary embodiment, the sets of raw data stored on the data store server 110 may all have a common pattern reflective of financial trades. In this exemplary embodiment, the feature engineering modules stored on the project store server 108 may all have features corresponding to this common pattern reflective of financial trades.

In yet another exemplary embodiment, sets of raw data stored on the data store server 110 may all have a common pattern reflective of empirical research data. In this exemplary embodiment, the feature engineering modules stored on the project store server 108 may all have features corresponding to this common pattern reflective of empirical research data.

As demonstrated by the exemplary embodiments, the sets of raw data having a common pattern or domain space and the feature engineering modules may span multiple domains of interest while remaining within the scope of the present disclosure.

Once the user has selected appropriate feature engineering modules, the method 200 continues in step 206 with the submission to the search engine 106 of feature engineering models included in the submission for a new AI project and in the existing AI projects selected from the project store server 108.

Once the feature engineering models have been submitted, the method 200 continues in step 208 with the forming of a search space by the search engine 106 comprising all the configuration parameters included in the submitted feature engineering modules. The search space reflects the common pattern of the raw data set from which it is derived and the customization of the user's selection of feature engineering modules.

Once the search space has been formed, the method 200 continues in step 210, with defining by the search engine 106 of a set of candidate configuration parameters selected from within the search space. As with the search space, the selected set of candidate parameters reflects a common pattern of the raw data sets from which it is derived and the customization of the user's selection of feature engineering modules.

As disclosed above, the search engine 106 may implement a grid search, a random search, or any other type of search known to a person of ordinary skill on the search space to define a set of candidate configuration parameters.

Once the candidate configuration parameters have been defined, the method 200 continues in step 212 with the feature selection module 106A defining appropriate features from the submitted feature engineering modules.

As disclosed above, the feature selection module 106A may implement a forward feature selection method, a backward feature elimination method, or any other known method to define the appropriate features from the combined features within the selected feature engineering modules.

As with the selected candidate parameters, the defined appropriate features reflect a common pattern of the raw data sets from which they are derived and the customization of the user's selection of feature engineering modules.

Once the appropriate features have been defined, the method 200 continues in step 214 with the submission of the appropriate features and the candidate configuration parameters to the model training and validation module 104A of the AI project server 104.

The model training and validation module 104A implements a model fitting by applying the candidate configuration parameters to the nodes within a neural network for a new AI project. A learning algorithm implemented by the model training and validation module 104A configures data inputs into the neural network based on the appropriate features.

The data elements used by the model training and validation module 104A may be selected from the data sets stored on the data store servers 110.

In addition to the candidate configuration parameters, the model training and validation module 104A may also apply hyperparameters to the neural network that have been provided by the search engine 106 to the neural network. The hyperparameters are variables that govern the training process itself as implemented by the learning algorithm. For example, a hyperparameter may define how many hidden layers of nodes to use between an input layer and an output layer of the neural network and how many nodes each layer should have. These variables are not directly related to the data sets stored on the data store server 110. Instead, they may be configuration variables that define the specific implementation of the neural network.

Once the data elements have been applied to the input layer of the neural network configured with the candidate configuration parameters, the method continues in step 216 with the gathering by the model training and validation module 104A of resulting data from the neural network's output layer.

Once the resulting data has been gathered, the method 200 continues in step 218 with transmitting by the AI project server 104 of the resulting data to the search engine 106.

Once the resulting data has been received, the method 200 continues in step 220 with determining by the search engine 106 of whether or not the neural network configured with the candidate configuration parameters converges based on the resulting data received from the AI project store 104.

Convergence may be determined when the difference between the value of the resulting data and a target data value falls within a predetermined error range. If convergence is found, the neural network configured with the candidate configuration parameters will provide an acceptable model.

If it is determined that there is convergence with the candidate configuration parameters, the method 200 continues in step 222 with the AI project server 104 creating a trained model based on the neural network configured with the candidate configuration parameters.

Once the model has been created, the method 200 ends in step 224 with sending by the AI project server 104 of the trained model to the model inference module 104B for serving the trained model and to the project store server 108 for storing the trained model. The trained model may be stored on the project storage server 108 along with its corresponding feature engineering module, configuration parameters, and text-based project description.

Alternatively, if convergence does not exist, the method 200 in step 226 returns to step 208 for defining by the search engine 106 an alternate set of configuration parameters selected from within the search pool and determining if convergence exists based on the alternate set of configuration parameters.

As with the original set of candidate parameters, the search engine 106 may implement a grid search, a random search, or any other type of search on the search space to define an alternate set of candidate parameters.

The foregoing description discloses only example embodiments. Modifications of the above-disclosed assemblies and methods which fall within the scope of this disclosure will be readily apparent to those of ordinary skill in the art.

This disclosure is not intended to limit the invention to the particular assemblies and/or methods disclosed, but, to the contrary, the intention is to cover all modifications, equivalents, and alternatives falling within the scope of the claims.

What is claimed is:

1. A system for implementing a method for the automated development of artificial intelligence (AI) projects comprising:
a project store server providing for the storage, searching, and retrieval of existing AI projects;
a data store server providing for the storage, searching, and retrieval of data sets;
a processor-based search engine executing instructions that implement a feature selection module providing for manual or automated selection of features applied to neural networks as part of the existing AI projects; and
a processor-based AI project server executing instructions that implement a model training and validation module, a model inference module, and an input module.

2. The system for implementing a method for the automated development of AI projects of claim 1 further comprising an external feature store providing model training services over the communication network, including predefined feature engineering modules.

3. The system for implementing a method for the automated development of AI projects of claim 2 further comprising a communication network electronically interconnecting the project store server, the data store server, the processor-based search engine, the processor-based AI project server, and the external feature store.

4. The system for implementing a method for the automated development of AI projects of claim 1 wherein each of the existing AI projects stored on the project store server includes a detailed description of the AI project.

5. The system for implementing a method for the automated development of AI projects of claim 4 wherein each of the existing AI projects stored on the project store server further includes accompanying models, feature engineering modules, and configuration parameters.

6. The system for implementing a method for the automated development of AI projects of claim 1 wherein the datasets stored on the data store server have a common pattern.

7. The system for implementing a method for the automated development of AI projects of claim 1 wherein the feature selection module implements a forward feature selection method, a backward feature elimination method, or any other selection method known to one of ordinary skill in the art.

8. The system for implementing a method for the automated development of AI projects of claim 1 wherein the search engine further executes instructions that implement a grid search, random search, Bayesian search, or any other standard search method known to a person of ordinary skill in the art.

9. The system for implementing a method for the automated development of AI projects of claim 1 wherein the model training and validation module provides for the creation, training, and validation of neural network-based models within the new AI projects.

10. The system for implementing a method for the automated development of AI projects of claim 9 wherein this model training and validation module may implement a cross-validation method for the training and validation of the neural network-based models within new AI projects.

11. The system for implementing a method for the automated development of AI projects of claim 9 wherein the model inference module provides for serving over the communications network of the neural network-based models that have been trained.

12. The system for implementing a method for the automated development of AI projects of claim 1 wherein the input module provides for receiving user inputs directly or over the communication network.

13. The system for implementing a method for the automated development of AI projects of claim 12 wherein the input module provides a graphical user interface over the communication network.

14. A method for implementing the automated development of AI projects, the steps comprising:
receiving at an input module a submission for a new AI project;
receiving at a project store server a selection of relevant existing AI projects;
submission to a search engine of submitted feature engineering modules included in the submission for a new AI project and the existing AI projects selected from the project store server;
forming by the search engine of a search space that comprises all configuration parameters included in the submitted feature engineering modules;
defining by the search engine of a set of candidate configuration parameters selected from within the search space;
defining by the feature selection module of appropriate features from the submitted feature engineering modules;
submitting the appropriate features and the candidate configuration parameters to a model training and validation module of an AI project server;
gathering by the model training and validation module of resulting data from the neural network's output layer;
transmitting by the AI project server of the resulting data to the search engine;
determining by the search engine of whether or not the neural network configured with the candidate configuration parameters converges based on the resulting data received from the AI project store server;
if there is convergence, creating by the AI project server of a trained model based on the neural network configured with the candidate configuration parameters and sending the trained model to the model inference module 104B for serving the trained model and to the project store server 108 for storing the trained model; and
if convergence does not exist, defining by the search engine of an alternate set of configuration parameters selected from within the search pool and determining if convergence exists based on the alternate set of configuration parameters.

15. The method for implementing the automated development of AI projects of claim 14 wherein the submission for the new AI project includes a feature engineering module.

16. The method for implementing the automated development of AI projects of claim 14 wherein the submission for the new AI project includes a text-based description that may be used to select existing AI projects.

17. The method for implementing the automated development of AI projects of claim 15 wherein the feature engineering module includes functions implementing preprocessing steps necessary to transform raw data into features for a machine-learning algorithm for the new AI project.

18. The method for implementing the automated development of AI projects of claim 15 wherein the feature engineering module includes a listing of variables derived from a raw data set, these variables being the inputs to the machine learning algorithm for the AI project.

19. The method for implementing the automated development of AI projects of claim 15 wherein the feature engineering module includes configuration parameters that define the weights and biases associated with inputs to each node within a neural network.
